(19) United States
(12) Patent Application Publication
(10) Pub. No.: US 2011/0295077 A1
(43) Pub. Date: Dec. 1, 2011

(54) Methods and Devices for Providing Multiple Devices Access to a Surgical Site Through a Single Port

(75) Inventors: David Stefanchik, Morrow, OH (US); William B. Weisenburgh, II, Maineville, OH (US); James T. Spivey, Cincinnati, OH (US)

(73) Assignee: Ethicon Endo-Surgery, Inc., Cincinnati, OH (US)

(21) Appl. No.: 12/790,370

(22) Filed: May 28, 2010

(51) Int. Cl. A61B 1/32

(52) U.S. Cl. 600/210

(57) ABSTRACT

Methods and devices are provided to assist in providing multiple devices access to a surgical site through a single port. In one exemplary embodiment an access device includes a body having a plurality of ports. The ports can include at least one primary instrument port and at least one secondary instrument port. A self-sealing passageway can be disposed between the primary and secondary instrument ports to allow communication between the two ports. As a result, a surgical instrument can be repositioned from one port to another without removing the instrument from the access device. In one embodiment a combined end effector nominal width of surgical instruments disposed in the ports can be greater than a diameter of the primary instrument port and/or a diameter of the body of the access device. Exemplary systems and methods for performing procedures using multiple devices in a single surgical opening are also provided.

### FIELD

The present invention relates to methods and devices for accessing a surgical site, and more particularly to methods and devices for introducing multiple instruments or devices into a body cavity through a single incision.

### BACKGROUND

Minimally invasive surgical techniques such as endoscopies and laparoscopies are often preferred over traditional open surgeries because the recovery time, pain, and surgery-related complications are typically less with minimally invasive surgical techniques. In many laparoscopic procedures, the abdominal wall is pierced and a cannula or trocar that is approximately 5 to 10 mm in diameter is inserted into the abdominal cavity. The abdominal cavity is insufflated with carbon dioxide gas to a pressure of approximately 15 mm Hg. Multiple cannulas or trocars can be inserted and directed to the surgical site so that multiple instruments with different functions can be used at the same time. While miniaturized versions of laparoscopic procedures have also been developed, the instruments for such procedures are generally more expensive and fragile, and still typically require the use of multiple instruments or channels that have diameters of about 2 to 3 mm.

Because of the rise in popularity of minimally invasive surgeries, there has been significant development with respect to the procedures and the instruments used in such procedures. For example, in some procedures a single incision at the navel can be sufficient to provide access to a surgical site. This is because the umbilicus can be a preferred way to access the abdominal cavity in a laparoscopic procedure. The umbilical incision can be easily enlarged without significantly compromising cosmesis and without significantly increasing the chances of wound complications, thus allowing multiple instruments to be introduced through a single incision.

One potential drawback to using a single incision to access a surgical site is that a device disposed in the incision to seal the outside environment from the surgical site must be configured to receive each instrument that will be used at the surgical site. Often it is desirable to have multiple instruments located at the surgical site at one time. When multiple instruments are placed through the device disposed in the incision, however, a port of the device configured to receive the multiple instruments must be configured to receive each of the instruments being used at the surgical site at the same time. As a result, the port must be large enough to enable it to receive each of the instruments as well as the end effectors of the instruments, which tend to be larger than the shafts of the instruments. The port must not only be large enough to receive these instruments, but it also must be large enough to allow the instruments to be moved and used all while allowing a surgeon to have precise control over each of the instruments. A consequence of having such relatively large ports is that the device itself must be large, often requiring a larger incision.

Accordingly, there is a need for improved methods and devices for accessing a surgical site that allow multiple devices or instruments to be disposed in a single access device for use at a surgical site. The methods and devices should not only reduce the amount of interference between the devices or instruments, thereby improving a surgeon's ability to control the devices or instruments, but they should also allow the access device to be as small as possible, to reduce the size of the incision used to access the surgical site and any resulting scarring that occurs as a result.

### SUMMARY

Methods and devices are generally provided to improve surgical procedures that use multiple devices or instruments that are disposed through a single incision to access a surgical site. In one embodiment an access device is in the form of a body, which can be made of an elastomeric material. The body includes a primary instrument port, a plurality of secondary instruments ports disposed adjacent to the primary instrument port, and at least one self-sealing passageway communicating between the primary instrument port and one of the secondary instrument ports. The primary port can be configured to enable a surgical instrument to pass therethrough. The secondary ports can have diameters that are less than the diameter of the primary port. The self-sealing passageway can allow a shaft of a surgical instrument to be repositioned between the primary instrument port and one of the secondary instrument ports without removing the surgical instrument from the access device. In one embodiment there is a self-sealing passageway associated with each secondary port such that each passageway allows communication between the primary port and the respective secondary port. In another embodiment there can be at least one secondary self-sealing passageway that allows communication between two secondary instrument ports. The secondary self-sealing passageway can allow a surgical instrument to be repositioned between the first and second secondary instrument ports without removing the surgical instrument from the access device and without using the primary instrument port as an intermediary port.

The body of the device can be capable of housing surgical instruments that have end effectors located at their distal ends. The primary instrument port can have a diameter that is sufficient to enable passage of an end effector of a surgical instrument to pass through the port. Each of the secondary instrument ports can have a diameter that is less than a width of the end effector, thereby preventing the end effector from passing through the secondary instrument ports. At least one of the primary instrument port and the secondary instrument ports can be configured to provide a fluid-tight seal when a surgical instrument is disposed therein. Further, at least one of the primary instrument port and the secondary instrument ports can be configured to provide a fluid-tight seal when no surgical instrument is disposed therein. In one embodiment a combined end effector nominal width of surgical instruments disposed in the body is greater than a diameter of the body. In another embodiment a combined end effector nominal width of the surgical instruments disposed in the device is greater than a diameter of the primary instrument port.

The access device can also include a housing within which at least a portion of the access device can be disposed. The resulting device can include a proximal end of the housing that is configured to be located outside of a patient, a distal end of the device that can be configured to be disposed in a body cavity underlying tissue, and an intermediate portion of the device extending between the proximal and distal ends and having the primary and secondary ports disposed therethrough.

In another embodiment of an access device, the device is in the form of a body, which can be made of an elastomeric material. The body includes at least one primary instrument port, a plurality of secondary instrument ports that are positioned adjacent to the primary instrument port, and a plurality of self-sealing channels. Each of the self-sealing channels can extend between the primary instrument port and one of the secondary instrument ports. The primary instrument port can have a diameter that is sufficient to enable passage of an end effector of a surgical instrument to pass through the primary port. Each of the secondary instrument ports can have a diameter that is insufficient to enable passage of the end effector of the surgical instrument to pass through the secondary port. The secondary instrument ports can be configured and dimensioned to form a fluid-tight seal around a shaft of a surgical instrument that is positioned therein. The self-sealing channels can be effective to enable movement of a surgical instrument between the primary instrument port and each of the secondary instrument ports without removing the surgical instrument from the access device. In one embodiment each channel can allow for communication between one of the primary instrument ports and one of the secondary instrument ports. Optionally, there can be at least one secondary self-sealing channel that allows for communication between two secondary instrument ports. The secondary self-sealing channel can allow a surgical instrument to be repositioned between the two secondary instrument ports without removing the surgical instrument from the access device and without passing the instrument through the primary instrument port to move from one secondary port to another secondary port. The primary instrument port and/or the secondary instrument ports can be configured and dimensioned to form a fluid-tight seal around a shaft of a surgical instrument positioned therein.

In one embodiment the access device includes surgical instruments. In such an embodiment, the body of the device can be configured such that the diameter of the body of the device can be less than the combined end effector width of the surgical instruments disposed therein. Alternatively, the body of the device can be configured such that the diameter of at least one of the primary instrument ports can be less than the combined end effect width of the surgical instruments disposed within the device. In one embodiment the diameter of the primary instrument port is in the range of about 1 millimeters to about 20 millimeters. In another embodiment the diameter of the secondary instrument port is in the range of about 0.5 millimeters to about 10 millimeters. The device can also include a retractor. In such an embodiment, the body can be disposed in a central portion of the retractor.

In yet another embodiment of an access device, the device is in the form of a body. The body can be made of an elastomeric material. The body includes a primary instrument port, at least one secondary instruments port disposed adjacent to the primary instrument port, and a self-sealing passageway communicating between the primary instrument port and the secondary instrument port. The primary port can be configured to enable a surgical instrument to pass therethrough. The secondary port can have diameters that is equal to or less than the diameter of the primary port. The self-sealing passageway can allow a shaft of a surgical instrument to be repositioned between the primary instrument port and the secondary instrument port without removing the surgical instrument from the access device. In one embodiment the at least one secondary instrument port is a plurality of secondary instrument ports and the device also includes additional self-sealing channels extending between the primary instrument port and one of the secondary instrument ports. In one embodiment there is a self-sealing passageway associated with each secondary port such that each passageway allows communication between the primary port and the respective secondary port. In another embodiment there can be at least one secondary self-sealing passageway that allows communication between two secondary instrument ports. The secondary self-sealing passageway can allow a surgical instrument to be repositioned between the first and second secondary instrument ports without removing the surgical instrument from the access device and without using the primary instrument port as an intermediary port.

In one embodiment of a surgical system, the system includes a plurality of surgical instruments and an access device. The instruments each have an elongate shaft and an end effector at a distal end thereof. The access device has a primary instrument access port, a plurality of secondary instrument access ports, and a self-sealing channel extending between the primary instrument access port and each of the secondary instrument access ports formed therein. The diameter of each of the secondary instrument ports can be less than the diameter of the primary instrument port. The end effectors of the surgical instruments are able to pass through the primary instrument port but are unable to pass through at least one of the secondary access ports. Each of the secondary instruments ports, however, can be configured to receive the shaft of the surgical instrument in a fluid-tight sealing arrangement. In one embodiment the diameter of the access device is less than the combined end effector width of the surgical instruments disposed therein. In another embodiment the diameter of the primary instrument access port is less than the combined end effector width of the surgical instruments disposed in the access device. For example, the diameter of the primary instrument access port can be in the range of about 1 millimeters to about 20 millimeters. The diameter of each of the secondary instrument ports can be in the range of about 0.5 millimeters to about 10 millimeters.

In another embodiment of a surgical system, the system includes a plurality of surgical instruments and an access device. The instruments each have an elongate shaft and an end effector at a distal end thereof. The access device has a primary instrument access port, at least one secondary instrument access port, and a self-sealing channel extending between the primary instrument access port and the secondary instrument access port formed therein. The diameter the secondary instrument port can be equal to or less than the diameter of the primary instrument port. The end effectors of the surgical instruments are able to pass through the primary instrument port but are unable to pass through the secondary access port. The secondary instruments port, however, can be configured to receive the shaft of the surgical instrument in a fluid-tight sealing arrangement. In one embodiment the diameter of the access device is less than the combined end effector width of the surgical instruments disposed therein. In another embodiment the diameter of the primary instrument access port is less than the combined end effector width of the surgical instruments disposed in the access device. For example, the diameter of the primary instrument access port can be in the range of about 1 millimeters to about 20 millimeters. The diameter of the secondary instrument port can be in the range of about 0.5 millimeters to about 10 millimeters. In one embodiment the system can include a plurality of secondary instrument access ports and additional sealing channels. Each additional sealing channel can extend between the primary instrument access port and one of the secondary instrument access ports.

In one embodiment of a surgical method, a retractor is positioned through an opening in tissue. The retractor includes a seal having a plurality of ports disposed therein, including a primary port and multiple secondary ports. A first surgical instrument is inserted through the primary port. The surgical instrument can be moved from the primary port to one of the secondary ports. Movement from the primary port to the secondary port can be accomplished without removing the surgical instrument from the seal. Additional surgical instruments can be inserted through the primary port. In one embodiment at least one additional surgical instrument is moved from the primary port to a secondary port without removing the instrument from the seal. Moving surgical instruments from the primary port to one of the secondary ports can include moving the respective instrument through a self-sealing passageway disposed between the primary and secondary ports. The method can also include moving one of the surgical instruments from one secondary port to an adjacent secondary port without removing the instrument from the seal and/or without moving the instrument into the primary port to then move it to the adjacent secondary port. Moving surgical instruments between secondary ports can include moving the instrument through a secondary self-sealing passageway disposed between the secondary port in which the instrument is positioned and the adjacent secondary port.

In one embodiment each of the surgical instruments have end effectors disposed on a distal end thereof. The diameters of the secondary ports can be insufficient to receive one of the end effectors. The combined end effector nominal width of the surgical instruments disposed in the ports can be greater than the diameter of the retractor. Alternatively, the combined end effector nominal width of the surgical instruments disposed in the ports can be greater than the diameter of the seal. Still further, a combined end effector nominal width of the surgical instruments disposed in the ports can be greater than the diameter of the primary port.

### BRIEF DESCRIPTION OF DRAWINGS

This invention will be more fully understood from the following detailed description taken in conjunction with the accompanying drawings, in which:

FIG. 1 is a top perspective view of one exemplary embodiment of an access device;

FIG. 2 is top perspective, cross-sectional view of the access device of FIG. 1 taken along line 2-2;

FIG. 3 is side, cross-sectional view of the access device of FIG. 1 taken along line 3-3;

FIG. 4 is a bottom perspective view of the access device of FIG. 1;

FIG. 5 is a bottom view of the access device of FIG. 4;

FIG. 6 is a bottom perspective, cross-sectional view of the access device of FIG. 4 taken along the line 6-6;

FIG. 7 is a top perspective view of another embodiment of an access device;

FIG. 8 is a side view of the access device of FIG. 7;

FIG. 9 is a top view of the access device of FIG. 7;

FIG. 10 is a top perspective, cross-sectional view of the access device of FIG. 9 taken along the line 10-10;

FIG. 11 is a perspective view of another embodiment of an access device;

FIG. 12 is a top view of the access device of FIG. 11;

FIG. 13 is a top view of yet another embodiment of an access device;

FIG. 14 is a top view of still another embodiment of an access device;

FIG. 15A is top perspective view of an access device similar to the access device of FIG. 1 having a first instrument disposed in a primary instrument port;

FIG. 15B is a top perspective view of the access device of FIG. 15A in which the first instrument is disposed in a secondary instrument port;

FIG. 15C is a top perspective view of the access device of FIG. 15B having a second instrument disposed in the primary instrument port;

FIG. 15D is a top perspective view of the access device of FIG. 15C in which the second instrument is disposed in another secondary instrument port;

FIG. 15E is a top perspective view of the access device of FIG. 15D in which the second instrument is disposed in a secondary instrument port that is adjacent to the another secondary instrument port;

FIG. 15F is a top perspective view of the access device of FIG. 15E in which the second instrument is disposed in a secondary instrument port adjacent to the adjacent secondary instrument port, a third instrument is disposed in the primary instrument port, and a fourth instrument is disposed in the first secondary instrument port;

FIG. 16 is a perspective view of still another embodiment of an access device, the access device being disposed in a retractor;

FIG. 17 is perspective view of the access device of FIG. 16, wherein a plurality of surgical instruments are disposed therethrough; and

FIG. 18 is a perspective view of another embodiment of a surgical access device having a housing.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, a person skilled in the art will also recognize that to the extent that the dimensions disclosed herein are described with respect to a diameter that is typically used to describe circular or cylindrical elements, equivalent measurements for components that are a different geometric shape can be easily determined. Likewise, a person skilled in the art will recognize that to the extent particular materials are discussed as having particular properties, such as being rigid or flexible, other materials can also be used, and further, materials can be treated in particular ways to make them more rigid or more flexible as desired.

A surgical access device is generally provided for minimally-invasive surgeries such as laparoscopic surgeries. The surgical access device can have a body and its body can be disposed in a body of a patient to allow for access to a surgical site from outside of the patient's body. The device can generally be configured to receive one or more instruments or tools through the device so that the instruments can be used to perform a desired procedure. Instruments can be received through one or more instrument ports of the device. An instrument disposed in the primary port can be moved from the primary port to a secondary port, adjacent to the primary port, by passing the instrument through a passageway disposed between the primary and secondary ports. This configuration allows an instrument to be repositioned to a different port of the device without removing the instrument from the device. Typically a secondary port has a diameter that is smaller than that of a primary port but large enough to receive at least a portion, such as a shaft, of the instrument. The ports can form seals around instruments disposed therein, and also can be configured to form a seal even when no instrument is disposed therein.

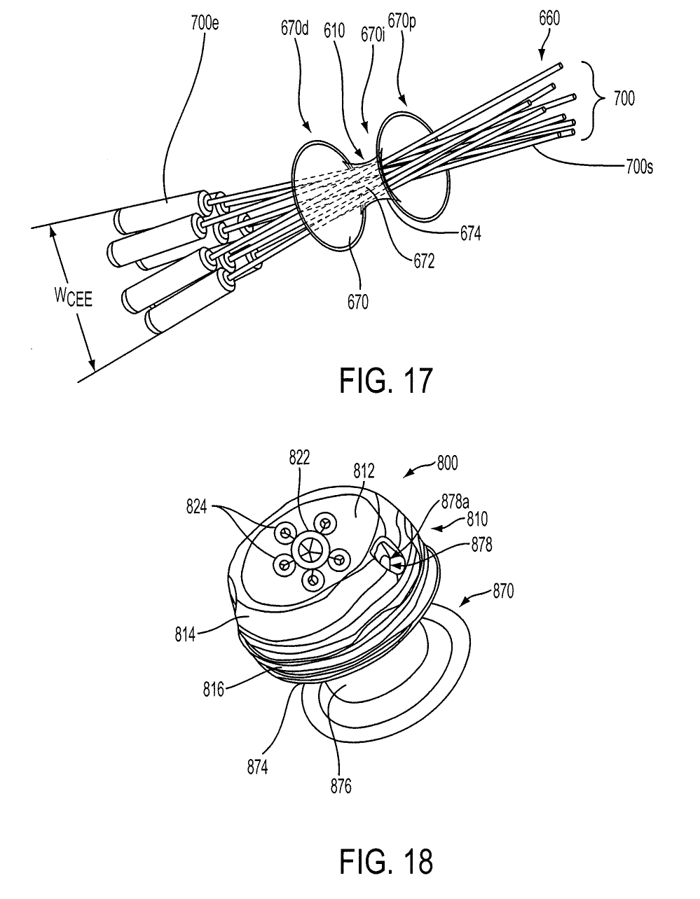

By moving an instrument from the primary port to a first secondary port, the primary port is available for the insertion of a second instrument. The second instrument can be disposed in the primary instrument and can also be moved from the primary port to a second secondary port. Any number of instruments can be disposed in the device provided the device has enough ports that are sized to receive at least a portion of the instruments. By allowing instruments to be moved from a primary instrument port to one or more secondary ports that are smaller than the primary port, the primary port can be used to first place an instrument within the device and then move the instrument to one of the secondary ports, which are typically sized to be too small to accept an end effector of an instrument. This configuration makes it possible to use instruments with shafts of smaller diameters and/or to use an access device with a smaller diameter, which makes it possible for surgeons to use smaller incisions. In fact, the net result can be a nominal width of the end effectors formed at the distal end of all of the tools able to be placed within the device that is greater than a diameter of at least the primary port, and even greater than the device in which the instruments are disposed. This combined end effector nominal width is the width formed by the respective edges of the two instruments disposed furthest apart from each other when disposed in the body of the device. One example of a combined end effector nominal width is illustrated in FIG. 17, where a combined end effector nominal width is labeled W_{CEE}.

Instruments that can be used in conjunction with the device can be of any type typically used in minimally invasive surgical procedures, such as laparoscopes. For the sake of discussion, instruments herein will generally be described as having a shaft and an end effector disposed at a distal end of the shaft, unless otherwise noted. The end effector is typically larger than the shaft, thereby enabling the end effector to pass through a larger port but have the instrument moved to a smaller port sized to receive the shaft of the instrument. By way of non-limiting example, some of the types of instruments that can be used in conjunction with the access device described include an endoscopic device, a tissue cutting device, a tissue grasping device, a fluid delivery device, an insufflation device, and a suturing device. Further, although the dimensions of a surgical instrument can be referred to by a number of different parameters, for ease of description purposes, end effectors are described herein as having a width and are generally illustrated as cylindrical or rectangular ends. End effectors, however, can have any shape and size. Thus, in other embodiments end effectors can have a diameter or any number of other appropriate dimensions.

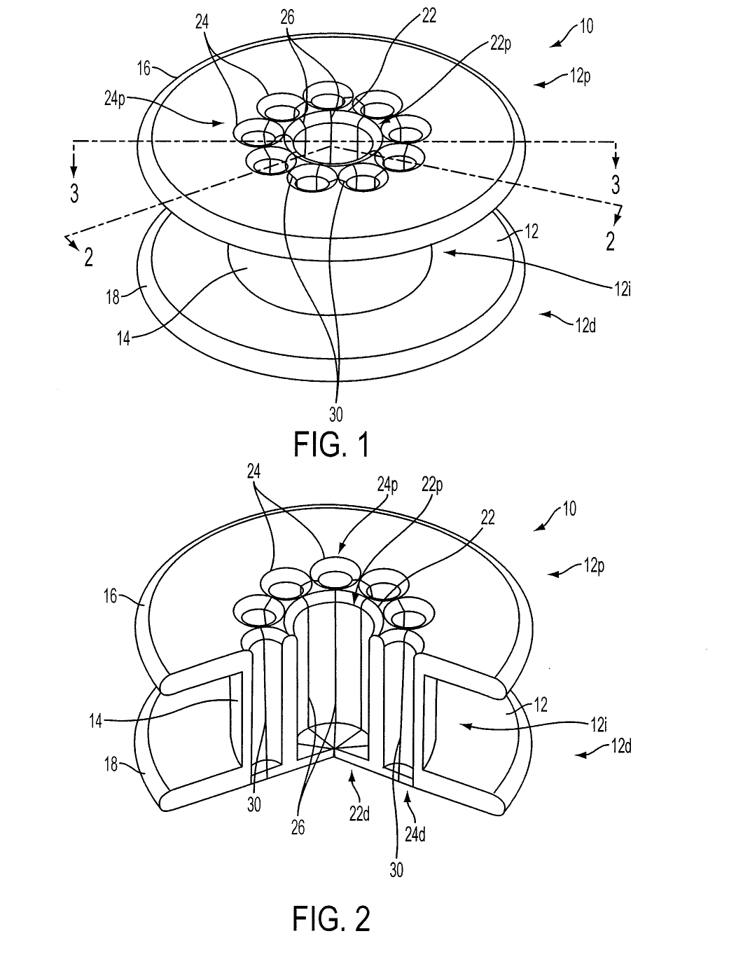

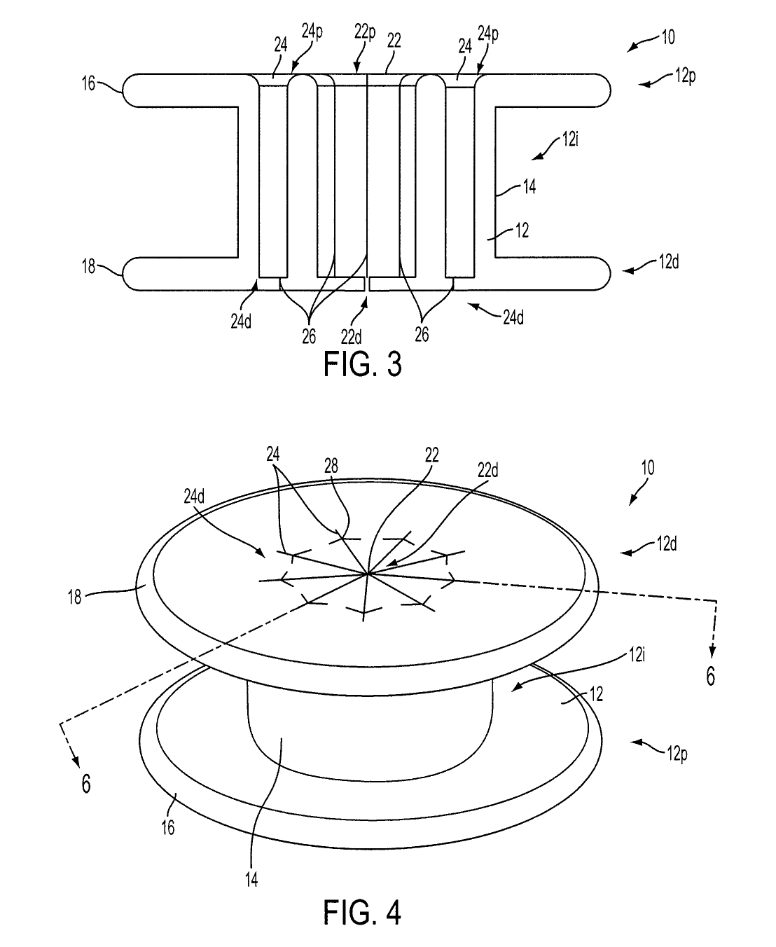

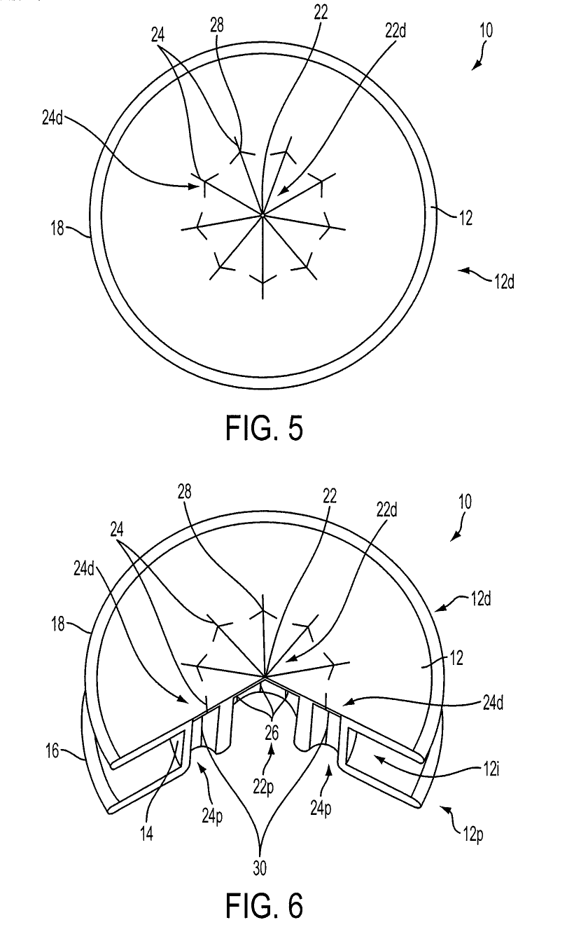

In one exemplary embodiment of an access device 10, as shown in FIGS. 1-6, the device 10 can have a body 12 configured to be disposed in an opening or incision of a patient's body, such as a natural orifice or a created orifice, for performing a surgical procedure, and further, can be configured to have one or more surgical instruments inserted therethrough. The body can include proximal and distal ends 12p, 12d with an intermediate portion 12i disposed therebetween. One or more access ports 22, 24 can be formed in the body such that the ports 22, 24 extend from the proximal end 12p, through the intermediate portion 12i, and into the distal end 12d. In the illustrated embodiment the access ports 22, 24 include a primary instrument access port 22 and nine secondary instrument access ports 24. Each of the access ports 22, 24 can be configured to allow access to a surgical site from an outside environment by positioning surgical instruments through the ports 22, 24. The device 10 can also include passageways 26, that are configured to allow for communication between the access ports. In the illustrated embodiment there is one primary passageway 26 and two secondary passageways 30 for each secondary port. Each primary passageway 26 is disposed between the primary port 22 and the respective secondary port 24 and each secondary passageway 30 is disposed between each of the two adjacent secondary ports 24. The primary passageway 26 can be configured to allow for an instrument to be moved between the primary port 22 and the secondary port 24 while maintaining a seal between the ports 22, 24 and between the outside environment and the surgical site. Likewise, the secondary passageway 30 can be configured to allow for an instrument to be moved between adjacent secondary ports 24 while maintaining a seal between the secondary ports 24 and between the outside environment and the surgical site.

The body 12 of the access device 10 can be configured to be positioned in an opening formed in tissue, and further, it can be configured to receive surgical instruments inserted therethrough. The body 12 can be generally flexible to assist in disposing the device in a surgical opening. In one exemplary embodiment the body 12 is made from an elastomeric material.

While the body 12 can be configured to have a variety of shapes, sizes, and configurations depending at least in part on the size of the opening in which the device will be disposed, additional components with which the device will be used, instruments that will be disposed in the body and its ports, and the type of surgical procedure with which the device will be used, the body 12 in the illustrated embodiment is generally cylindrical. More particularly, as shown, the body 12 includes a proximal flange 16 at the proximal end 12p, a distal flange 18 at the distal end 12d, and an inner elongate portion extending therebetween. The inner elongate portion can be bound by a sidewall 14. The inner elongate portion can have a diameter less than a diameter of the proximal and distal flanges 16, 18, which can have the same diameter or different diameters from one another. The proximal flange 16 can be configured to be seated on tissue of the patient adjacent to an outside environment to allow access to a surgical site from the outside environment. Alternatively, the proximal flange 16 can be configured to be seated within or coupled to a housing configured to be seated adjacent to a surgical opening, such as the housing recited in U.S. patent application Ser. No. 12/399,482 entitled “Methods and Devices for Providing Access into a Body Cavity” of Weisenburgh II, et al., and filed on Mar. 6, 2009, the contents of which is incorporated herein by reference. The distal flange 18 can be configured to be disposed in a body cavity underlying tissue to grasp tissue and maintain the location of the device 10 within the opening.

While the body can have a variety of sizes, in one exemplary embodiment, a diameter of both the proximal end 12p and the distal end 12d of the body 12 is in the range of about 40 millimeters to about 80 millimeters, and more specifically is about 60 millimeters. As a result of the present invention, a combined end effector nominal width of a plurality of surgical instruments that can be disposed in the body 12 can be greater than the diameter of the body 12, i.e., greater than about 40 millimeters, or about 60 millimeters, or even about 80 millimeters. In one exemplary embodiment the combined end effector nominal width can be about 1.25 times to about 2 times larger than a diameter of the body in which the instruments are disposed, and more specifically can be about 1.5 times larger than a diameter of the body in which the instruments are disposed.

The body 12 can be formed from a variety of flexible materials, such as polymeric materials. Exemplary materials that can be used to form the body include polyisoprene, polyurthethane, silicone, urethane, thermoplastic elastomer, and rubber. More than one material can be used to form the body 12, and the body 12 can include some portions that are more rigid than other portions. For example, the sidewalls 14 of the intermediate portion 12i can be more rigid than other portions of the device. Examples of more rigid materials that can be used include polycarbonate, polyester, polyetherimide, and stainless steel.

The plurality of ports 22, 24 that extend through the body 12 of the device 10 can be configured to receive instruments for use at the surgical site. The plurality of ports 22, 24 can be angled relative to a plane extending across the proximal end 12p of the body 12 and/or rotatable or other wise movable relative to the plane extending across the proximal end 12p of the body 12 and/or other portions(s) of the body 12. Any number of ports can be included in the body 12, but in the illustrated embodiment there are ten ports 22, 24. The ten ports 22, 24 include the primary port 22 and the nine secondary ports 24. In the illustrated embodiment the primary port 22 is centrally located and the secondary ports 24 are adjacent to and symmetrically disposed around the primary port 22. Further as shown, the primary port 22 has a diameter that is larger than a diameter of any of the secondary ports 24. This allows passage through the primary port 22 of instruments having end effectors that have widths that are larger than a diameter of a secondary port 24 but approximately equal to or smaller than a width of the primary port 22. Because the shafts of instruments are typically smaller in diameter than the end effectors, once the portion of the end effector that is larger than a secondary port 24 passes through the body 12, the instrument can be moved to a secondary port 24, for instance by passing the instrument across a passageway 26 disposed between the primary and secondary ports 22, 24, thereby freeing the primary port 22 to receive further instruments.

A proximal end 22p, 24p of the ports 22, 24 can be configured to receive at least a portion of an instrument. In the illustrated embodiment, the proximal end 22p of the primary instrument port 22 is configured to receive an end effector and a shaft of an instrument, while a proximal end 24p of the secondary instrument ports 24 is configured to receive only a shaft of the same instrument. A distal end 22d, 24d of the ports 22, 24 can include seals 28 that are configured to form a seal between the surgical site and the device 10, and thereby the surgical site and the outside environment. As shown particularly in FIGS. 4-6, each of the distal ends 22d, 24d of the ports 22, 24 forms a fluid-tight seal when no instrument is disposed in the port 22, 24. Although virtually any type of seal can be used, the seal of the illustrated embodiment is a “star-seal.” The seals 28 can also be configured to form a fluid-tight seal around instruments disposed in the ports 22, 24. When an instrument is removed from the port 22, 24, the seal 28 can maintain the fluid-tight seal by returning to its configuration prior to any instrument being inserted therein. The seals 28, thus, can be self-sealing. The sealing features disposed in the distal end 12d of the body 12 of the illustrated embodiment can also be incorporated into the proximal end 12p of the body 12, in lieu of, or in addition to, the sealing features in the distal end 12d. Instruments can still be disposed through seals formed in the proximal end 12p, and thus such an embodiment would still allow the proximal end 12p to be configured to receive at least a portion of an instrument. Those skilled in the art will appreciate that different types of seals can be disposed at the proximal and distal ends 12p, 12d of the body 12.

Similar to the body 12, the size, shape, and configuration of the ports 22, 24 will also depend, at least in part, on the size of the opening in which the device will be disposed, additional components with which the device will be used, instruments that will be disposed in the body and its ports, and the type of surgical procedure with which the device will be used. For example, the primary port 22 can be disposed in a non-central location. By way of further non-limiting example, there can be multiple primary ports that are sized larger than one or more secondary ports. Likewise, the secondary ports 24 can have a variety of shapes, sizes, and configurations. For example, specific secondary ports can be shaped and sized to receive instruments having different shaft or even end effector shapes and diameters. In one embodiment the primary and/or secondary ports can be configured to receive multiple instruments in the same port. Because the ports 22, 24 are part of the body 12, typically the ports 22, 24 will be made out of the same materials as the body 12. Thus, the ports 22, 24 can be generally flexible. In some embodiments it can be desirable to form the ports 22, 24 from a material that is different than at least a portion of the body 12. For example, the ports 22, 24 can be made more rigid than the proximal and distal flanges 16,18.

In one exemplary embodiment, a diameter of the primary instrument port 22 can be in the range of about 1 millimeter to about 20 millimeters, and more specifically can be about 10 millimeters, and a diameter of the secondary instrument ports 24 can be in the range of about 0.5 millimeters to about 10 millimeters, and more specifically can be about 3 millimeters. In such a configuration, instruments having shafts and/or end effectors as large as about 20 millimeters can be disposed in the primary port 22 and instruments having shafts and/or end effectors as large as about 10 millimeters can be disposed in the secondary ports 24. As a result of the present invention, a combined end effector nominal width of a plurality of surgical instruments that can be disposed in the body 12 can be greater than the diameter of the primary port 22, i.e., greater than about 1 millimeter, greater than about 10 millimeters, or even greater than about 20 millimeters. In one exemplary embodiment the combined end effector nominal width can be about two times to about ten times larger than a diameter of the primary instrument port 22 of the body 12 in which the instruments are disposed, and more specifically can be about four times larger than a diameter of the primary instrument port 22 of the body 12 in which the instruments are disposed. Further, in embodiments in which the ports 22, 24 are made of a flexible materials, portions of a tool that are slightly larger than a diameter of the ports 22, 24 can be received by the ports 22, 24 because of the flexible nature of the ports 22, 24. This slight expansion of the size of the diameters of the ports 22, 24 can be achieved without losing a fluid-tight seal between the instruments and the ports 22, 24 and between the surgical site and the outside environment.

The passageways 26, or channels, disposed between the primary port 22 and the secondary ports 24 can allow for communication between the primary port 22 and one or more of the secondary ports 24. The passageways 26 can be self-sealing. Accordingly, when an instrument is not being passed through the passageway 26, a fluid-tight seal can be formed between the ports 22, 24 that the passageway 26 is disposed between and between the surgical site and the outside environment. When an instrument is disposed in the passageway 26, the passageway 26 can be configured to form a seal around the instrument, thereby maintaining a fluid tight-seal between the ports 22, 24 and between the surgical site and the outside environment. As shown, each secondary port 24 has its own passageway 26 disposed between the primary port 22 and the secondary port 24.

In an alternative embodiment, one or more passageways 30 or channels can optionally be formed between two secondary ports 24. This can allow for instruments to be moved from one secondary port 24 to a second secondary port 24 without using the primary port 22 as an intermediary port and without removing the instrument from the body 12. The passageways 30 can be configured in a manner similar to the passageways 26 formed between the primary instrument port 22 and the secondary instrument ports 24. Because the passageways 26, 30 are part of the body 12, typically the passageways 26, 30 will be made out of the same materials that the body 12 is made. Thus, the passageways 26, 30 can be generally flexible. In some embodiments it can be desirable to form the passageways 26, 30 out of a material that is different than at least a portion of the body 12. For example, the materials used to create one or more of the self-sealing passageways 26, 30 can be more flexible than the materials used to form the sidewall 14 of the body 12.

Any number, size, and configuration of passageways 26 or channels can be used. For example, not every secondary port 24 must have a passageway 26 disposed between the secondary port 24 and the primary port 22. Secondary ports 24 can be isolated for receiving an instrument, or alternatively, can have a passageway 30 formed between an adjacent secondary port. One skilled in the art will appreciate that any number of paths can be formed between any number of primary ports 22 and secondary ports 24 formed in the body 12 of the present device 10.

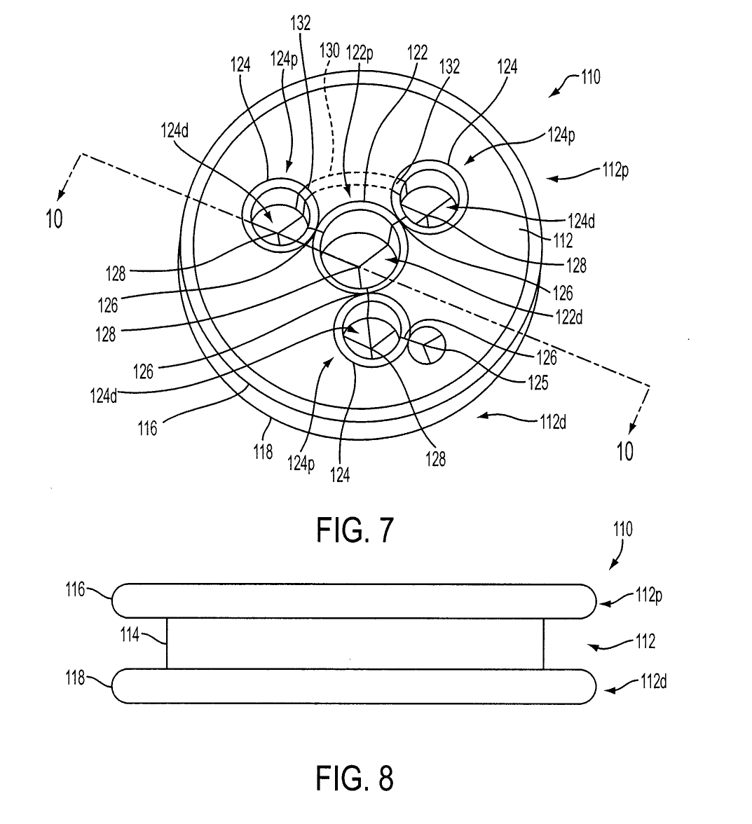

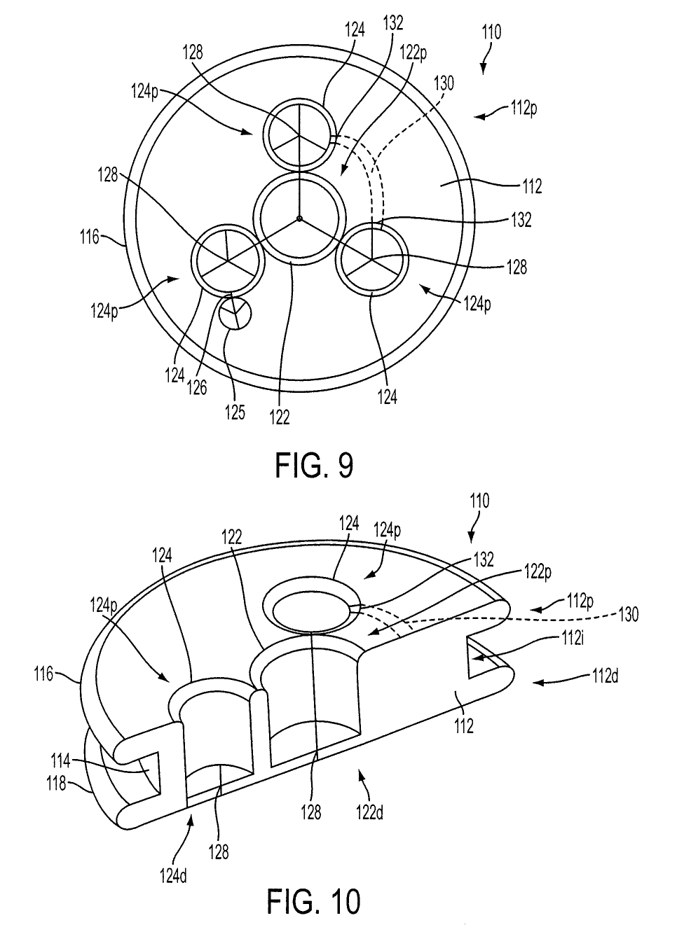

Another embodiment of an access device 110 is illustrated in FIGS. 7-10. The device 110 has many of the same properties as the device 10 of FIGS. 1-6, but it includes fewer secondary ports 124. The device 110 as shown has a body 112 configured to be disposed in an opening for performing a surgical procedure, proximal and distal ends 112p, 112d that each include a flange 116, 118, respectively, and an intermediate portion 112i disposed between the proximal and distal ends 112p, 112d. The intermediate portion 112i can include an inner elongate portion, as shown a sidewall 114, through which a plurality of ports 122, 124 can be formed. In the illustrated embodiment, a single primary port 122 is located centrally on the body 112, three secondary ports 124 are disposed adjacent to the primary port 122 such that the secondary ports 124 are radially equidistant from each other, and a tertiary port 125 is adjacent to one of the secondary ports 124. The tertiary port 125 can have features similar to the features disclosed with respect to ports generally herein and can have any shape, size, configuration, etc. In the illustrated embodiment the tertiary port 125 is smaller than the secondary port 124, but in other embodiments it can be equal to or even larger than the secondary port 124 and it can be located in any portion of the body 112. An opening at a proximal end 122p, 124p, 125p of each of the ports 122, 124, 125 respectively, can be configured to receive an instrument, and a seal 128 can be disposed at a distal end 122d, 124d, 125d of each of the ports 122, 124, 125 respectively, thereby creating a fluid-tight seal between the surgical site and an outside environment. As shown, passageways 126 can be disposed between the primary port 122 and each of the secondary ports 124, as well as between secondary ports 124 and the tertiary port 125. In alternative embodiments, only some of the secondary ports 124 can have passageways 126 and/or one or more passageways 130 or channels can be disposed between adjacent secondary ports 124, as illustrated by the dashed lines in FIGS. 7, 9, and 10. Entry into the passageways 130 in the illustrated embodiment can be controlled by fluid-tight sealing channel doors 132. The same types of properties and various configurations, shapes, sizes, and materials that were described with respect to the device 10 of FIGS. 1-6 are equally applicable to the device 110 of FIGS. 7-10.

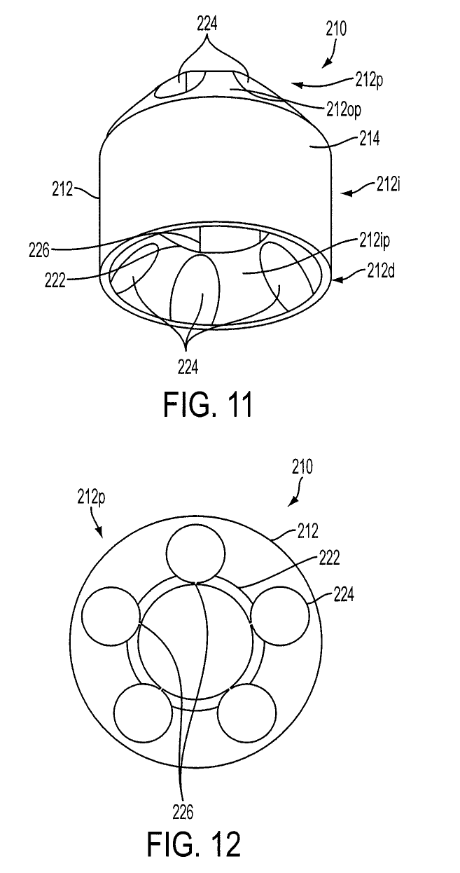

Another embodiment of an access device 210 is illustrated in FIGS. 11 and 12. Similar to the devices 10, 110 illustrated in FIGS. 1-10, the device 210 of FIGS. 11 and 12 includes a body 212 having a plurality of ports 222, 224 formed therein. The ports 222, 224 include a primary instrument port 222 that is centrally located and multiple secondary ports 224 that are adjacent to and in communication with the primary port 222. In the illustrated embodiment there are five secondary instrument ports 224. A passageway 226 can be disposed between the primary port 222 and each of the secondary instrument ports 224. The ports 222, 224 and passageways 226 can have the same properties and configurations as discussed with respect to the ports 22, 24, 122, 124 and passageways 26, 126 of the devices 10, 110 of FIGS. 1-10, including, by way of non-limiting example, passageways or channels formed between adjacent secondary instrument ports and distal ends of the ports that are configured to seal both when an instrument is disposed in the ports and when no instrument is disposed in the ports.

Unlike the devices 10, 110 of FIGS. 1-10, the body 212 of the device 210 of FIGS. 11 and 12 does not include flanges. Instead, an outer portion 212o of a proximal end 212p of the body 212 is tapered such that it has a progressively decreasing diameter moving towards a proximal end of the device 210. Further, an inner portion 212i of a distal end 212d of the body 212 can also be tapered. As illustrated, the inner portion 212i is tapered such that it has a progressively increasing diameter moving towards a distal end of the device 210. The plurality of ports 222, 224 can be formed in the intermediate portion 212i. As shown, a sidewall 214 of the intermediate portion 212i can be the largest width of the body 212. The body 212 can be configured to be disposed in a surgical opening, or it can be configured to be used in conjunction with other devices such that it is disposed in the other devices. Likewise, although in the illustrated embodiment the outer portion 212o and the inner portion 212i of the body 212 is tapered, the body 212 can have any number of shapes and can include tapers in other directions or no tapers at all of either or both of the outer portion 212o and the inner portion 212i.

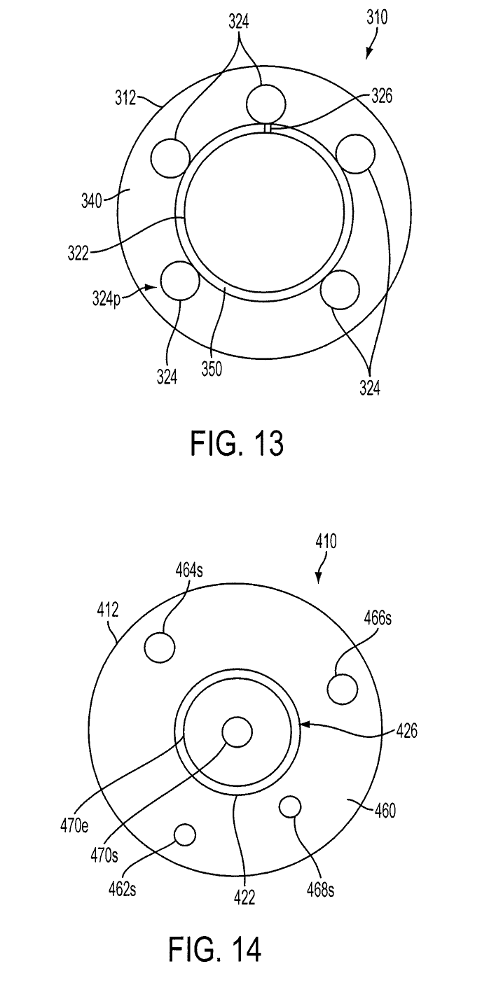

In yet another embodiment of an access device 310 for accessing a surgical site from an outside environment, which is illustrated in FIG. 13, a single passageway 326 or channel can be formed that can be used to reposition a surgical instrument from a primary instrument port 322 to a plurality of secondary instrument ports 324 of a body 312. As shown, the body 312 includes a port ring 340 in which each secondary port 324 is disposed. The ports 322, 324 can be configured in a similar manner as described with respect to ports 22, 24, 122, 124, 222, 224 in FIGS. 1-12, and thus proximal ends 322p, 324p of the ports 322, 324 can be openings that are configured to receive at least a portion of a surgical instrument therein and distal ends 322d, 324d can be configured to seal both when an instrument is disposed in the ports 322, 324 and when no instrument is disposed in the ports 322, 324. The port ring 340 can be configured to rotate, thereby allowing each of the secondary ports 324 to become aligned with the passageway 326 that allows the primary port 322 to communicate with each of the secondary ports 324.

Each of the secondary ports 324 can be configured such that when the port 324 aligns with the passageway 326, an instrument can be repositioned from the primary port 322 to the secondary port 324, or from the secondary port 324 to the primary port 322, by way of the passageway 326. When the secondary ports 324 are not aligned with the passageway 326, or when one of the secondary ports 324 is aligned with the passageway 326 but an instrument is not being repositioned between the primary port 322 and one of the secondary ports 324, the secondary ports 324 can remain sealed, thereby preventing any leakage of fluid. Even when the instrument is moved from the primary port 322, through the passageway 326, and into the aligned secondary port 324, the seal can be maintained around the instrument. Thus, the passageway 326 can be self-sealing such that it remains sealed until both one of the secondary ports 324 is aligned with the passageway 326 and an instrument is moved from the primary port 322, in contact with and then through the passageway 326, and into the aligned secondary port 324.

Alternatively, the port ring 340 in which the secondary ports 324 are disposed can be immobile, and instead a passageway ring 350 in which the passageway 326 is formed can be configured to rotate. When an instrument is to be repositioned from the primary port 322 to one of the secondary ports 324, the passageway ring 350 can be rotated into alignment with the desired secondary port 324. The configuration between the primary port 322, the passageway 326, and each of the secondary ports 324 can be the same as discussed with respect to the rotating port ring 340, thereby enabling a seal to be maintained throughout the use of the device 310. In some embodiments both the port ring 340 and the passageway ring 350 can be rotatable. A seal can be formed between any of the port ring 340, the passageway ring 350, and any other components of the device 310 to prevent fluid leakage, for example by using one or more coatings. Although not illustrated, two or more secondary ports 324 can be configured to communicate between adjacent secondary ports 324, in manners similar to those discussed with respect to the secondary ports 24, 124, 224 of the devices 10, 110, 210 of FIGS. 1-12.

As a result of the configurations discussed with respect to FIG. 13, while any number of passageways or channels can be used, the number of passageways or channels can be less than the number of secondary ports while still allowing the primary port to communicate with each of the secondary ports. This configuration in which a single passageway 326 can be configured to be used with multiple secondary ports 324 can be incorporated into any configuration of the devices disclosed herein.

While the devices of FIGS. 1-13 include predefined ports, in another embodiment, illustrated in FIG. 14, a body 412 of an access device 410 can be formed of a puncturable, self-sealing material 460. In one exemplary embodiment the material 460 has a durometer of at least about 5 Shore A and has a plurality of pores formed therein such that the pore volume in the sealing material 460 is at least about one percent, as discussed in greater detail in U.S. patent application Ser. No. 12/479,293 entitled “Methods and Devices for Providing Access Through Tissue to Surgical Site” of Hess et al., and filed on Jun. 5, 2009, which is hereby incorporated by reference in its entirety.

As shown, a primary instrument port 422 is formed in the body 412 and is surrounded by the puncturable, self-sealing material 460. A self-sealing passageway 426 or channel can be formed at an edge of the primary port 422 and can be configured to allow instruments to be repositioned from the primary port 422 and into the self-sealing material 460 while maintaining a seal around the instrument, and thus between a surgical site and an outside environment. Once the instrument is fully disposed in the self-sealing material 460, the passageway 426 can close, thereby maintaining a fluid-tight seal between the primary instrument port 422 and the self-sealing material 460. A shaft of the instrument can then be located in a desired location in the self-sealing material 460, the self-sealing material 460 serving a similar purpose as the secondary instrument ports discussed above. As a result, a plurality of instruments can be disposed in the body 412 of the device 410 such that a combined end effector nominal width can be greater than a diameter of the primary instrument port 422 and/or a diameter of the body 412 itself, similar to the other devices disclosed herein. In the illustrated embodiment, four instrument shafts 462s, 464s, 466s, and 468s are shown as being disposed in the self-sealing material 460 and a fifth instrument end effector 470e, with its respective shaft 470s of the instrument, is shown disposed in the primary port 422.

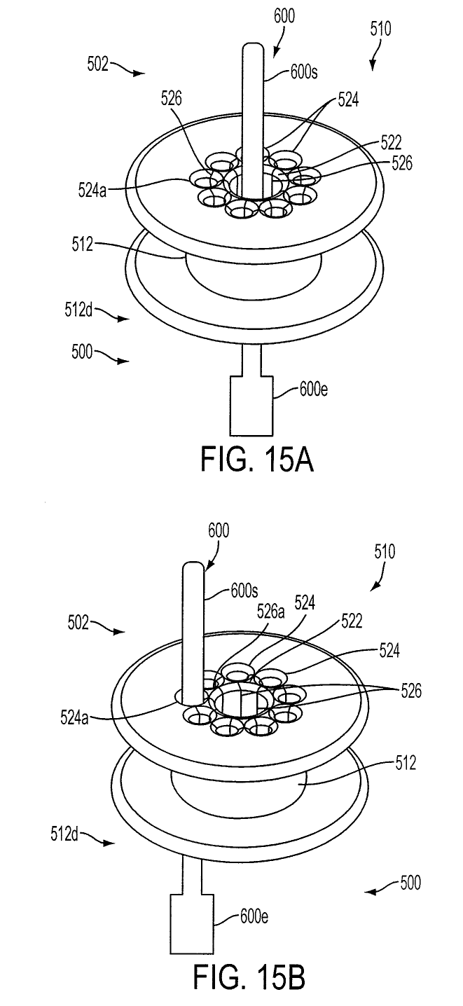

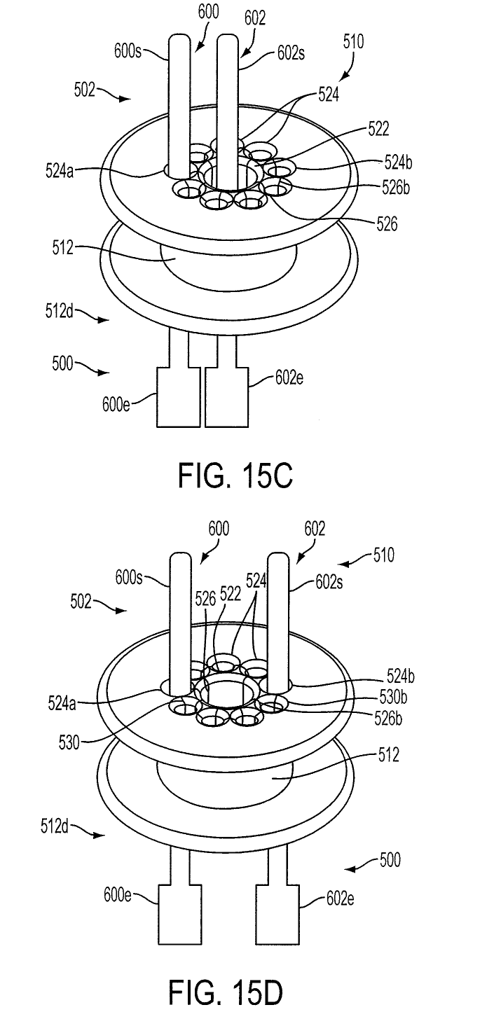

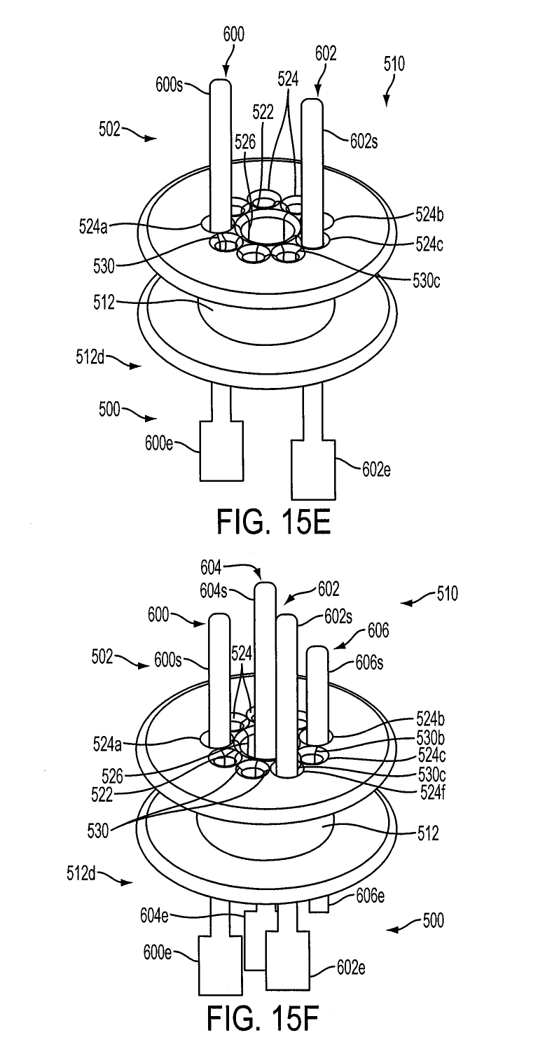

In use, as shown in FIGS. 15A-15F, devices in accordance with the present invention can be used to allow multiple instruments to be disposed through a single opening to access a surgical site. As a result of the configuration of a body of the device, a combined end effector nominal width of the instruments that can be housed within the device is greater than a diameter of a primary instrument port or even greater than a diameter of a body of the device. As shown in FIG. 15A, a device having a body 512, in this instance a retractor 510 configured like the device 10 of FIGS. 1-6, is disposed in a surgical opening to access a surgical site 500. The retractor 510 includes a plurality of ports 522, 524 and passageways 526, 530. The retractor 510 forms a seal between an outside environment 502 and the surgical site 500. A first instrument 600 is disposed in a primary instrument port 522 of the body 512 of the retractor 510. A width of an end effector 600e of the first instrument 600 can be greater than a diameter of secondary ports 524, but can be approximately equal to or less than a diameter of the primary port 522. A width of a shaft 600s of the first instrument 600 can be approximately equal to or less than a diameter of the secondary port 524, and thus is less than a diameter of the primary port 522.

Once the end effector 600e clears a distal end 512d of the body 512, the first instrument 600 can be moved from the primary port 522 to a first secondary port 524a of the plurality of secondary ports 524, as shown in FIG. 15B. To do so, the first instrument 600 passes through a first primary passageway 526a disposed between the primary port 522 and the first secondary port 524a. The first primary passageway 526a can be self-sealing, and thus the passageway 526a can form a seal around the shaft 600s of the first instrument 600 as it passes therethrough. Likewise, the first secondary port 524a can be self-sealing, and thus the first secondary port 524a can form a seal around the shaft 600s of the first instrument 600 disposed therein.

A second instrument 602 can be disposed through the primary port 522, as shown in FIG. 15C. Similar to the first instrument 600, a width of an end effector 602e of the second instrument 602 can be larger than a diameter of the secondary ports 524 but approximately equal to or less than a diameter of the primary port 522, and a width of a shaft 602s of the second instrument 602 can be approximately equal to or less than a diameter of the secondary ports 524, and thus is less than a diameter of the primary port 522. Once the end effector 602e of the second instrument 602 clears the distal end 512d of the body 512, the second instrument 602 can be moved from the primary port 522 to a second secondary port 524b, as shown in FIG. 15D. The second instrument 602 passes through a second primary passageway 526b disposed between the primary port 522 and the second secondary port 524b. The second primary passageway 526b can be self-sealing, and thus the passageway 526b can form a seal around the shaft 602s of the second instrument 602 as it passes therethrough. Likewise, the second secondary port 524b can be self-sealing, and thus the second secondary port 524b can form a seal around the shaft 602s of the second instrument 602 disposed therein.

As shown in FIGS. 15D and 15E, in embodiments in which secondary passageways 530 or channels are formed between the secondary ports 524, an instrument can be moved from one secondary port 524b to another secondary port 524c. As illustrated, the second instrument 602 is moved from the second secondary port 524b to the third secondary port 524c, which is adjacent to the second secondary port 524b. A first secondary passageway 530b is disposed between the second and third secondary ports 524b, 524c, and the first secondary passageway 530b and third secondary port 524c can form seals like the aforementioned passageways and secondary ports.

The second instrument 602 can be moved to still another secondary port, fourth secondary port 524f, by way of a second secondary passageway 530c formed between the third secondary port 524c and the fourth secondary port 524f. As shown in FIG. 15F, the second instrument 602 is moved closer to the first instrument 600. Further, a third instrument 604 can be introduced into the primary port 522. A width of an end effector 604e of the third instrument 604 can be approximately equal to or less than the diameter of the primary instrument port 522 and a shaft 604s of the third instrument 604 can also be approximately equal to or less than the diameter of the primary instrument 522. If it is desired to move the third instrument 604 into one of the plurality of secondary ports 524, then the shaft 604s can be approximately equal to or less than the diameter of one of the secondary ports 524 and less than the diameter of the primary instrument port 522. Still further, instruments can also be introduced directly through the secondary instruments ports 524. As shown in FIG. 15F, a fourth instrument 606 is disposed directly in the second secondary port 524b. The fourth instrument 606 has an end effector 606e that is approximately equal to or less than the diameter of the second secondary port 524b, and it likewise has a shaft 606s that is approximately equal to or less than the diameter of the second secondary port 524b. As illustrated, the shaft 606s and the end effector 606e of the fourth instrument 606 have approximately equal widths.

A person skilled in the art would recognize that any number of procedures can be preformed using the methods described herein. The order in which instruments are disposed and repositioned between the ports is practically unlimited, and will depend, at least in part, on the type of procedure being performed and the type of instruments or tools being used during the procedure. Further, although in the illustrated embodiment there is a large amount of space between the secondary ports 524 and a peripheral edge of the proximal end 512p of the body 512, in other embodiments the amount of space can be small. Thus, while in the illustrated embodiment it appears difficult to have a combined end effector nominal width that is greater than a diameter of the body 512, in other embodiments it can be quite easy to have a combined end effector nominal width that is greater than a diameter of the body 512. One such example is provided in FIG. 17.

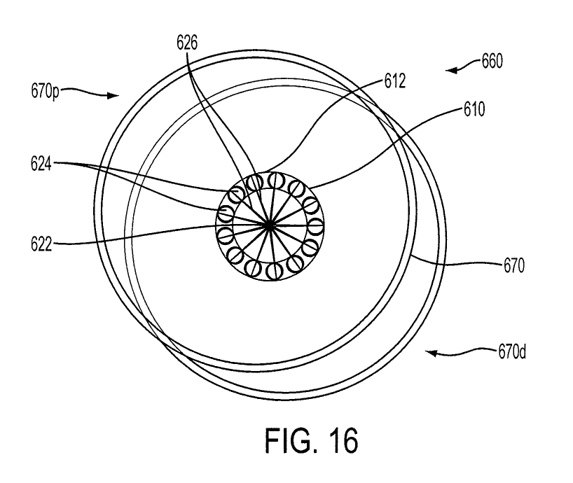

In one exemplary embodiment of a surgical access system, the system includes an access device and a plurality of surgical instruments. As shown in FIG. 16, an access device of a system 660 can be a seal 610 that includes a plurality of ports 622, 624 configured to receive surgical instruments of the system 660. A body 612 of the seal 610 can be cylindrical in shape and can have a uniform diameter throughout its proximal, intermediate, and distal portions 612p, 612i, and 612d. Other sizes, shapes, and configurations, however, can also be used. The plurality of ports 622, 624 of the seal 610 can include a primary instrument port 622 and one or more secondary instrument ports 624. In the illustrated embodiment there are thirteen secondary instrument ports 624 and they are disposed adjacent to and symmetrically around the primary instrument port 622 in a “star-seal” design. As shown in FIG. 17, the primary instrument port 622 can be configured to receive surgical instruments 700 having end effectors 700e that have widths that are greater than a diameter of any one of the secondary instrument ports 624. Each of the primary and secondary instruments ports 622, 624 can be self-sealing. Likewise, passageways 626 or channels disposed between the primary and secondary instruments ports 622, 624 can also be self-sealing. Accordingly, a seal can be maintained between a surgical site and an outside environment at all times, regardless of whether an instrument is disposed in the seal 610 or not. Although not illustrated, passageways or channels can also be formed between two or more secondary instrument ports 624, similar to the passageways 30, 130, 230, and 530 of the devices 10, 110, 210, and 510, respectively.

The seal 610 can be disposed in a retractor 670, which can optionally be part of the system 660, having proximal and distal ends 670p, 670d and an inner elongate portion, as shown a sidewall 674, forming a cavity disposed between the proximal and distal ends 670p, 670d. As shown, the seal 610 is disposed in a central portion of the retractor 670. The proximal end 670p of the retractor 670 can be configured to be seated on tissue of a patient adjacent to an outside environment to allow access to a surgical site from the outside environment. The distal end 670d can be configured to be disposed in a body cavity underlying tissue to grasp tissue and maintain the location of the retractor 670 within the opening. The inner elongate portion can have a diameter less than a diameter of the proximal and distal ends 670p, 670d, which can have the same diameter or different diameters from one another. The retractor 670 and the seal 610 can be separate, coupled components, or alternatively, they can be integrally formed.

The surgical instruments 700 of the system 660 can be disposed through the seal 610 by first inserting the instruments 700 through the primary port 622 and then repositioning the instruments 700 into one of the secondary ports 624, for instance, by moving an instrument 700 from the primary port 622, through the passageway 626, and into one of the secondary ports 624. The resulting configuration is one in which the combined end effector nominal width W_{CEE} is not only greater than a diameter of the primary port 622, but is also greater than a diameter of the seal 610 in which the ports 622, 624 are disposed and a diameter of the proximal and distal ends 670p, 670d of the retractor 670. In other embodiments, the system can include other components, such as other access devices that can be used interchangeably. Any number of access devices and surgical instruments can be included with the system to form a kit, including but not limited to the access devices and instruments disclosed herein. A housing configured to be located outside of the body, adjacent to a surgical opening and coupled to a device like the devices 10, 110 or the retractor 670, can also be included in the system. The access devices can then be removably and replaceably coupled to the housing, for instance by way of male and female components associated with the device and housing, a snap-fit configuration, an interference-fit configuration, or any number of ways by which two components can be coupled. One exemplary embodiment of a housing coupled to a device like the devices 10, 110 or the retractor 670 to form an access device 800 is illustrated in FIG. 18 and is described with more particularly in U.S. patent application Ser. No. 12/399,482 entitled “Methods and Devices for Providing Access into a Body Cavity” of Weisenburgh II, et al., and filed on Mar. 6, 2009, the contents of which was already incorporated herein by reference.

As shown, a housing 810 is configured to have one or more surgical instruments inserted therethrough. In the illustrated embodiment the housing 810 includes a proximal housing 814, a seal base 812 that supports at least one primary port 822 and at least one secondary port 824, and a distal housing 816. The seal base 812 can be configured in a manner similar to the proximal ends 12p and 112p of the devices 10 and 110 and the body 612 of the seal 610. The primary and secondary ports 822, 824 can be angled relative to the seal base 812 and/or rotatable or other wise movable relative to the seal base 812 and/or other portions(s) of the housing 814. The housing 810 can be removably coupled to a retractor 870 configured to distally extend from the housing 810 and to provide a pathway through tissue into a body cavity. In the illustrated embodiment, the retractor 870 includes a proximal retractor portion or proximal retractor base 874 coupled to a distal retractor portion 876. The housing 810 can be movable with respect to the retractor 870. Such a configuration can help facilitate instrument positioning in a body cavity to which the device 800 provides access. While in the illustrated embodiment the seal base 812 is configured to be separate from the retractor 870 disposed therein, in other embodiments the seal base 812 can be part of a proximal end of the retractor 870, similar to the embodiments described with respect to the devices 10 and 110.

The device can include an insufflation port 878 supported by the proximal housing 814, although a person skilled in the art will appreciate that the insufflation port 878 can be located elsewhere in the housing 810 or in other locations. A person skilled in the art will also appreciate that the insufflation port 878 can have a variety of configurations. Generally, the insufflation port 878 can be configured to pass an insufflation fluid through an insufflation orifice 878a of the insufflation port 878 through a flexible insufflation tube into and/or out of a body cavity to which the device 800 provides access.

The housing 810 of the surgical access device 800 can have a variety of configurations. As shown in this embodiment, the proximal housing 814 is in the form of a seal cap configured to releasably mate the seal base 812 to the retractor 870, and the distal housing 816 is in the form of an o-ring configured to be disposed between the seal base 812 and the retractor 870 to form a seat and seal between the base 812 and a distal portion of the device 800, e.g., the retractor 870. The retractor 870, the seal base 812, the proximal housing 814, and the distal housing 816 can each have various sizes, shapes, and configurations, depending at least in part on the size of the opening in which the device 800 will be disposed, additional components with which the device 800 will be used, instruments that will be disposed in the base 812 and its ports 822, 824, and the type of surgical procedure with which the device 800 will be used.

The devices, systems, and methods disclosed herein can be used on their own, or they can be used in conjunction with a variety of other devices. Likewise, the devices, systems, and methods disclosed herein can be incorporated into a variety of other devices. By way of non-limiting examples, the devices 10, 110, 210, 310, and 410, the retractor 510, the seal 610, and the teachings related to the same can be used on their own as a retractor, a seal, or as other types of access devices, including the types of devices referenced herein. Other types of surgical access devices with which the present inventions can be used include, but are not limited to, a wound protector, cannula, ring retractor, or other member for forming a pathway through tissue. The devices 10, 110, 210, 310, and 410, the retractor 510, the seal 610, and the teachings related to the same can also be used in various seal configurations, including, e.g., duckbill seals, cone seals, flapper valves, gel seals, diaphragm seals, lip seals, iris seals, etc. In one embodiment in which the device is a seal, the seal can be configured to be disposed in a central portion of a retractor, for example by an interference or coupled fit. Alternatively, the device can be associated with a housing configured to be located outside of the body, adjacent to a surgical opening. The device can then be removably and replaceably coupled to the housing, for instance by way of male and female components associated with the device and housing, a snap-fit configuration, an interference-fit configuration, or any number of ways by which two components can be coupled. The proximal end of the housing can be configured to be located outside of a patient, a distal end of the device can be disposed in a body cavity underlying tissue, and an intermediate portion extending between the proximal and distal ends can have primary and secondary ports disposed therethrough.

Examples of the types of devices, systems, and methods with which the teachings related to the devices, systems, and methods as described herein can be used and/or incorporated into include the access devices, systems, and methods disclosed in U.S. patent application Ser. No. 12/399,473 entitled “Methods and Devices for Providing Access into a Body Cavity” of Weisenburgh II, et al., and filed on Mar. 6, 2009, U.S. patent application Ser. No. 12/399,482 entitled “Methods and Devices for Providing Access into a Body Cavity” of Weisenburgh II, et al., and filed on Mar. 6, 2009, U.S. patent application Ser. No. 12/479,006 entitled “Interlocking Seal Components” of Widenhouse et al., and filed on Jun. 5, 2009, U.S. patent application Ser. No. 12/479,030 entitled “Retractor with Integrated Wound Closure” of Weisenburgh et al., and filed on Jun. 5, 2009, U.S. patent application Ser. No. 12/479,092 entitled Active Seal Components of Scheib et al., and filed on Jun. 5, 2009, U.S. patent application Ser. No. 12/512,542 entitled “Methods and Devices for Providing Access into a Body Cavity” of Martin et al., and filed on Jul. 30, 2009, and U.S. patent application Ser. No. 12/512,568 entitled “Methods and Devices for Providing Access into a Body Cavity” of Widenhouse et al., and filed on Jul. 30, 2009, each of which is hereby incorporated by reference in their entireties. Alternatively, the teachings related to ports that communicate between one or more passageways to allow a device disposed in one port to be moved to another port can be incorporated into a variety of devices, systems, and methods, including retractors, seals, and the devices, systems, and methods disclosed in the aforementioned references that were incorporated by reference in their entirety.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the devices described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and its contents are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A surgical method, comprising:
positioning a retractor through an opening in tissue, the retractor including a seal having a plurality of ports disposed therein;
inserting a first surgical instrument through a primary port of the plurality of ports;
moving the first surgical instrument from the primary port to a secondary port of the plurality of ports without removing the surgical instrument from the seal; and
inserting one or more additional surgical instruments through the primary port.

2. The method of claim 1, wherein the secondary port has a diameter that is insufficient to receive an end effector of a surgical instrument disposed therein.

3. The method of claim 1, wherein at least one surgical instrument of the one or more additional surgical instruments is moved from the primary port to another secondary port of the plurality of ports without removing the at least one surgical instrument from the seal.

4. The method of claim 1, wherein moving the first surgical instrument from the primary port to the secondary port further comprises moving the instrument through a self-sealing passageway disposed between the primary port and each of the secondary ports.

5. The method of claim 1, further comprising moving the first surgical instrument from one of the secondary ports to another of the secondary ports without removing the surgical instrument from the seal.

6. The method of claim 5, wherein moving the first surgical instrument from one of the secondary ports to the another of the secondary ports further comprises moving the instrument through a secondary self-sealing passageway disposed between the secondary ports.

7. The method of claim 1, wherein each of the surgical instruments has an end effector disposed on a distal end thereof, and a combined end effector nominal width of the surgical instruments, when disposed in the ports, is greater than a diameter of the retractor.

8. The method of claim 1, wherein each of the surgical instruments has an end effector disposed on a distal end thereof, and a combined end effector nominal width of the surgical instruments, when disposed in the ports, is greater than a diameter of the seal.

9. The method of claim 7, wherein each of the surgical instruments has an end effector disposed on a distal end thereof, and a combined end effector nominal width of the surgical instruments, when disposed in the ports, is greater than a diameter of the primary port.

10. A surgical access system, comprising:
a plurality of surgical instruments, each having an elongate shaft and an end effector at a distal end thereof; and
an access device having formed therein a primary instrument access port having a first diameter, a plurality of secondary instrument access ports each with a diameter less than the first diameter, and one or more self-sealing channels extending between the primary instrument access port and each of the secondary instrument access ports,
wherein the end effector of the surgical instruments is able to pass through the primary instrument access port but is unable to pass through at least one of the secondary instrument access ports and wherein each secondary instrument access port is configured to receive a shaft of the surgical instrument in a fluid-tight sealing arrangement.

11. The system of claim 10, wherein a diameter of the access device is less than a combined end effector width of the plurality of surgical instruments disposed therein.

12. The system of claim 11, wherein the first diameter is less than a combined end effector width of the plurality of surgical instruments disposed in the access device.

13. The system of claim 10, wherein the first diameter is in the range of about 1 millimeters to about 20 millimeters.

14. The system of claim 10, wherein the diameter of each of the secondary instrument access ports is in the range of about 0.5 millimeters to about 10 millimeters.

15. A surgical access system, comprising:
a plurality of surgical instruments, each having an elongate shaft and an end effector at a distal end thereof; and
an access device having formed therein a primary instrument access port having a first diameter, at least one secondary instrument access port having a diameter equal to or less than the first diameter, and a self-sealing channel extending between the primary instrument access port and the at least one secondary instrument access port,
wherein the end effector of the surgical instruments is able to pass through the primary instrument access port but is unable to pass through the at least one secondary instrument access port and wherein the at least one secondary instrument access port is configured to receive a shaft of the surgical instrument in a fluid-tight sealing arrangement.

16. The system of claim 15, wherein the at least one secondary instrument access port is a plurality of secondary instrument access ports, the system further comprising additional self-sealing channels, each of the additional self-sealing channels extending between the primary instrument access port and one of the plurality of secondary instrument access ports.

17. The system of claim 15, wherein a diameter of the access device is less than a combined end effector width of the plurality of surgical instruments disposed therein.

18. The system of claim 17, wherein the first diameter is less than a combined end effector width of the plurality of surgical instruments disposed in the access device.

19. The system of claim 15, wherein the first diameter is in the range of about 1 millimeters to about 20 millimeters.

20. The system of claim 15, wherein the diameter of the at least one secondary instrument access port is in the range of about 0.5 millimeters to about 10 millimeters.
